(19) United States
(12) Patent Application Publication
(10) Pub. No.: US 2015/0209384 A1
(43) Pub. Date: Jul. 30, 2015

(54) ORAL PREPARATION FOR PROMOTING EXPRESSION OF TGF-BETA, ORAL PREPARATION FOR INHIBITING PRODUCTION OF PAIN-MEDIATING SUBSTANCE, AND ORAL PREPARATION FOR PREVENTING EDEMA

(71) Applicant: KEWPIE CORPORATION, Tokyo (JP)

(72) Inventors: Hitoshi Kurihara, Fuchu-shi (JP); Takahiro Tsuruki, Fuchu-shi (JP); Akira Asari, Musashino-shi (JP)

(73) Assignee: KEWPIE CORPORATION, Tokyo (JP)

(21) Appl. No.: 14/382,154

(22) PCT Filed: Feb. 28, 2013
(86) PCT No.: PCT/JP2013/055431

§ 371 Date: Aug. 29, 2014

(30) Foreign Application Priority Data
Mar. 1, 2012 (JP) 2012-045954
Mar. 1, 2012 (JP) 2012-045955

(51) Int. Cl. A61K 31/728; A61K 9/00

(52) U.S. Cl. CPC A61K 31/728; A61K 9/0053

(57) ABSTRACT

An oral preparation for promoting expression of TGF-β includes as an active ingredient hyaluronic acid or a pharmaceutically acceptable salt thereof, the oral preparation promoting expression of TGF-β in plasma. In addition, an oral preparation for suppressing production of a pain-producing substance includes as an active ingredient hyaluronic acid or a pharmaceutically acceptable salt thereof, the oral preparation suppressing production of a pain-producing substance by promoting expression of TGF-β in plasma. In addition, an oral preparation for suppressing production of a pain-producing substance includes as an active ingredient hyaluronic acid or a pharmaceutically acceptable salt thereof, the oral preparation suppressing production of a pain-producing substance PGE2 and/or bradykinin. In addition, an oral preparation for suppressing edema includes as an active ingredient hyaluronic acid or a pharmaceutically acceptable salt thereof, the oral preparation suppressing production of a pain-producing substance PGE2 and/or bradykinin.

### TECHNICAL FIELD

The present invention relates to an oral preparation for promoting expression of TGF-β, an oral preparation for suppressing production of a pain-producing substance, and an oral preparation for suppressing edema.

### BACKGROUND ART

Hyaluronic acid, which is a mucopolysaccharide present in a living body, in particular, a subcutaneous tissue, has been widely utilized as a cosmetic raw material because of its high moisturizing function (Patent Literature 1). Hyaluronic acid has also been utilized as a pharmaceutical. For example, treatment has been performed for suppressing articular inflammation, involving compensating for a reduction in content of hyaluronic acid that a living body originally has by intra-articular injection of hyaluronic acid.

However, in the case of the intra-articular injection of hyaluronic acid, a patient needs to regularly visit a hospital to receive treatment, resulting in a heavy burden on the patient. In addition, local pain, swelling, or flare may occur at a site of the injection. Further, when articular pain is not so severe, it is considered that appropriate treatment including prevention is rarely performed.

### CITATION LIST

### Patent Literature

[Patent Literature 1]JP 63-57602 A

### SUMMARY OF INVENTION

### Technical Problem

The present invention provides an oral preparation for promoting expression of TGF-β, an oral preparation for suppressing production of a pain-producing substance, and an oral preparation for suppressing edema, which contribute to an improvement in QOL of a patient.

### Solution to Problem

The inventors of the invention of the present application have made extensive studies on pain (inflammatory pain), and as a result, have surprisingly found that hyaluronic acid or a pharmaceutically acceptable salt thereof promotes the expression of TGF-β in plasma. Thus, the present invention has been completed. In addition, the inventors of the invention of the present application have made intensive studies on pain (inflammatory pain), and as a result, have surprisingly found that hyaluronic acid or a pharmaceutically acceptable salt thereof suppresses the production of a pain-producing substance PGE2 and/or bradykinin. Thus, the present invention has been completed.

An oral preparation for promoting expression of TGF-β according to one embodiment of the present invention includes as an active ingredient hyaluronic acid or a pharmaceutically acceptable salt thereof, the oral preparation promoting expression of TGF-β in plasma.

An oral preparation for suppressing production of a pain-producing substance according to another embodiment of the present invention includes as an active ingredient hyaluronic acid or a pharmaceutically acceptable salt thereof, the oral preparation suppressing production of a pain-producing substance by promoting expression of TGF-β in plasma. In this case, the pain-producing substance may include bradykinin.

An oral preparation for suppressing edema according to still another embodiment of the present invention includes the oral preparation for promoting expression of TGF-β.

An oral preparation for suppressing production of a pain-producing substance according to still another embodiment of the present invention includes as an active ingredient hyaluronic acid or a pharmaceutically acceptable salt thereof, the oral preparation suppressing production of a pain-producing substance PGE2 and/or bradykinin.

An oral preparation for suppressing edema according to still another embodiment of the present invention includes as an active ingredient hyaluronic acid or a pharmaceutically acceptable salt thereof, the oral preparation suppressing production of a pain-producing substance PGE2 and/or bradykinin.

### Advantageous Effects of Invention

According to the oral preparation for promoting expression of TGF-β and the oral preparation for suppressing production of a pain-producing substance, the hyaluronic acid or the pharmaceutically acceptable salt thereof promotes the expression of TGF-β in plasma, thereby suppressing the production of the pain-producing substance, and hence pain can be relieved. In addition, according to the oral preparation for suppressing edema, edema can be suppressed by incorporating the oral preparation for promoting expression of TGF-β.

According to the oral preparation for suppressing production of a pain-producing substance, the hyaluronic acid or the pharmaceutically acceptable salt thereof suppresses the production of the pain-producing substance PGE2 and/or bradykinin, and hence pain can be relieved. In addition, according to the oral preparation for suppressing edema, edema can be suppressed by incorporating as the active ingredient the hyaluronic acid or the pharmaceutically acceptable salt thereof to suppress the production of the pain-producing substance PGE2 and/or bradykinin.

In addition, the oral preparation for promoting expression of TGF-β, the oral preparation for suppressing production of a pain-producing substance, and the oral preparation for suppressing edema are each an oral preparation, and hence can be easily ingested by a patient. Thus, the patient does not need to visit a hospital for treatment, which can contribute to an improvement in QOL of the patient. Accordingly, a burden on the patient can be reduced and pain can be relieved.

### BRIEF DESCRIPTION OF DRAWINGS

FIG. 1 show photographs, each showing the state of a left hind paw 6 hours after carrageenan induction in a test for confirming the pain suppressing action of hyaluronic acid of Example 1 of the present invention.

FIG. 2 is a graph showing time-dependent changes in paw volume of left hind paws by carrageenan induction in the test for confirming the pain suppressing action of hyaluronic acid of Example 1 of the present invention.

FIG. 3 is a graph showing the amounts of exudates 0 hours, 3 hours, and 6 hours after carrageenan induction in the test for confirming the pain suppressing action of hyaluronic acid of Example 1 of the present invention.

FIG. 4 is a graph showing the concentrations of TGF-β1 in plasma 0 hours, 3 hours, and 6 hours after carrageenan induction in the test for confirming the pain suppressing action of hyaluronic acid of Example 1 of the present invention.

FIG. 5 is a graph showing the total amounts of bradykinin in exudates 0 hours, 3 hours, and 6 hours after carrageenan induction in the test for confirming the pain suppressing action of hyaluronic acid of Example 1 of the present invention.

FIG. 6 is a graph showing the concentrations of bradykinin in exudates 0 hours, 3 hours, and 6 hours after carrageenan induction in the test for confirming the pain suppressing action of hyaluronic acid of Example 1 of the present invention.

FIG. 7 is a graph showing the total amounts of prostaglandin E2 (PGE2) 0 hours, 3 hours, and 6 hours after carrageenan induction in the test for confirming the pain suppressing action of hyaluronic acid of Example 1 of the present invention.

FIG. 8 is a graph showing the concentrations of PGE2 in exudates 0 hours, 3 hours, and 6 hours after carrageenan induction in the test for confirming the pain suppressing action of hyaluronic acid of Example 1 of the present invention.

FIG. 9 is a graph showing the measurement results of the concentrations of hyaluronic acid in plasma in the test for confirming the pain suppressing action of hyaluronic acid of Example 1 of the present invention.

FIG. 10 is a view schematically illustrating the action mechanism of an oral preparation for promoting expression of TGF-β of the present invention.

FIG. 11 is a graph showing the measurement results of TGF-β mRNA expression amounts for evaluating the TGF-β expression promoting action of hyaluronic acid in human colon-derived HT29 cells in Example 2 of the present invention.

### DESCRIPTION OF EMBODIMENTS

The present invention is hereinafter described in detail with reference to the drawings. It should be noted that in the present invention, “part(s)” means “part(s) by mass” and “%” means “mass %” unless otherwise specified.

### 1. ORAL PREPARATION FOR PROMOTING EXPRESSION OF TGF-β

An oral preparation for promoting expression of TGF-β according to one embodiment of the present invention contains as an active ingredient hyaluronic acid or a pharmaceutically acceptable salt thereof, and promotes the expression of TGF-β in plasma.

1.1. Hyaluronic Acid or Pharmaceutically Acceptable Salt Thereof

In the present invention, the “hyaluronic acid” refers to a polysaccharide having one or more repeating constituent units each formed of a disaccharide of β-D-glucuronic acid and β-D-N-acetylglucosamine. That is, the hyaluronic acid is a di- or higher polysaccharide including at least one disaccharide unit in which position 1 of β-D-glucuronic acid and position 3 of β-D-N-acetyl-glucosamine are bound to each other. In addition, examples of the “pharmaceutically acceptable salt of hyaluronic acid” include, but are not particularly limited to, a sodium salt, a potassium salt, a calcium salt, a zinc salt, a magnesium salt, and an ammonium salt.

The hyaluronic acid or the pharmaceutically acceptable salt thereof may be one extracted from a biological tissue (e.g., cock's comb, umbilical cord, skin, or synovial fluid) of an animal or the like. Alternatively, there may be used, for example, one obtained by culturing microorganisms, animal cells, or plant cells (e.g., a fermentation method involving using a Streptococcus bacterium or the like) or one synthesized chemically or enzymatically.

It should be noted that the purity of the hyaluronic acid or the pharmaceutically acceptable salt thereof to be used in the present invention only needs to be at a level that allows pharmaceutical use, and may be preferably 90% or more, more preferably 95% or more. This purity is a value calculated from a glucuronic acid determination value measured by a carbazole-sulfuric acid method (e.g., the Japanese Pharmacopoeia).

The carbazole-sulfuric acid method is a method involving measuring the absorbance (530 nm) of a sample solution obtained by: adding a hyaluronic acid aqueous solution into a sodium borate/sulfuric acid solution, followed by mixing; decomposing hyaluronic acid by heating, followed by cooling; adding a carbazole/ethanol solution, followed by mixing; and heating the mixture, followed by cooling. A calibration curve using similarly treated D-glucuronolactone is prepared to calculate a value in terms of D-glucuronolactone, and then the value is multiplied by 1.102 to determine a glucuronic acid determination value. The resultant glucuronic acid determination value is multiplied by (molecular weight of hyaluronic acid/molecular weight of glucuronic acid) to calculate the content of the hyaluronic acid.

In addition, the average molecular weight of the hyaluronic acid or the pharmaceutically acceptable salt thereof to be used in the oral preparation for promoting expression of TGF-β according to this embodiment is preferably 500,000 or more, more preferably 600,000 or more, still more preferably from 600,000 to 1,600,000. When the average molecular weight of the hyaluronic acid or the pharmaceutically acceptable salt thereof is less than 500,000, it becomes difficult to promote the expression of TGF-β in some cases. In addition, when the average molecular weight of the hyaluronic acid and/or the salt thereof is more than 1,600,000, the hyaluronic acid and/or the salt thereof has difficulty in dissolving, and its effect cannot be sufficiently exhibited in some cases.

It should be noted that the average molecular weight of the hyaluronic acid or the pharmaceutically acceptable salt thereof defined in the present invention is measured by the following method.

That is, a solution obtained by accurately weighing about 0.05 g of purified hyaluronic acid and dissolving the hyaluronic acid in a sodium chloride solution having a concentration of 0.2 mol/L to make exactly 100 mL, and solutions obtained by exactly weighing 8 mL, 12 mL, and 16 mL of the above-mentioned solution and adding a sodium chloride solution having a concentration of 0.2 mol/L to the respective solutions to make exactly 20 mL are used as sample solutions. In accordance with Viscosity Determination (Method I Viscosity measurement by capillary tube viscometer) in General Tests, Processes and Apparatus of the Japanese Pharmacopoeia (14th Edition), each of the sample solutions and the sodium chloride solution having a concentration of 0.2 mol/L are measured for its specific viscosity at 30.0±0.1C (Equation (1)), and a reduced viscosity is calculated at each concentration (Equation (2)). A graph is drawn by taking the reduced viscosity on the ordinate axis and the concentration of a test substance in terms of dry matter (g/100 mL) on the abscissa axis, and a limiting viscosity is determined from the intersection of a straight line connecting respective points with the ordinate axis. The limiting viscosity thus determined is substituted into Laurent's equation (Equation (3)) to calculate an average molecular weight (T. C. Laurent, M. Ryan, A. Pietruszkiewicz: B. B. A., 42, 476-485 (1960)).

Specific viscosity={(Number of seconds required for flowing of sample solution)/(Number of seconds required for flowing of 0.2 mol/L sodium chloride solution)}−1 (Equation 1)

Reduced viscosity=Specific viscosity/(Concentration of test substance in terms of dry matter (g/100 mL)) (Equation 2)

Limiting viscosity=3.6×10⁻⁴M^{0.78} (Equation 3)

M: average molecular weight

The content of the hyaluronic acid or the pharmaceutically acceptable salt thereof in the oral preparation for promoting expression of TGF-β according to this embodiment only needs to be an amount that allows the hyaluronic acid or the pharmaceutically acceptable salt thereof to function as the active ingredient, and is generally 1 mass % or more, preferably from 5 to 95 mass %.

1.2. Promotion of Expression of TGF-β

The oral preparation for promoting expression of TGF-β according to this embodiment promotes the expression of TGF-β in plasma of humans or non-human animals. In addition, the oral preparation for promoting expression of TGF-β according to this embodiment promotes the expression of TGF-β in cells or tissues of humans or non-human animals.

The promotion of the expression of TGF-β in plasma of and cells or tissues of humans or non-human animals may be confirmed, for example, by a known biochemical analysis method such as the detection or determination of TGF-β mRNA by northern blotting, a DNA array, a DNA chip, or the like, or the detection or determination of a TGF-β protein by western blotting, ELISA, affinity chromatography, or the like.

Examples of the non-human animals include mammals including non-primates (e.g., cattle, swine, horses, dogs, cats, rats, and mice) and primates (e.g., monkeys). The humans or the non-human animals are preferably humans.

It has been revealed that transforming growth factor-β (TGF-β) is one kind of cytokine (secretory protein that regulates cell functions), and has five subtypes β1 to β5. In addition, TGF-β is known to produce an extracellular matrix protein, suppress a degrading enzyme, and promote wound healing, in a number of tissues, and is also known to promote the growth and neogenesis of epithelial cells.

When the oral preparation for promoting expression of TGF-β according to this embodiment is orally ingested by humans or non-human animals, the expression of TGF-β in plasma can be suppressed in the humans or the non-human animals. As a result of this, pain can be relieved in patients (humans or non-human animals) suffering from inflammatory diseases (e.g., pathological conditions selected from the group consisting of: rheumatoid arthritis (RA); asthma; allergic diseases such as rhinitis; vascular diseases; thrombosis or harmful platelet aggregation; reocclusion after thrombolysis; reperfusion injury; inflammatory skin diseases such as psoriasis, eczema, contact dermatitis, and atopic dermatitis; diabetes (e.g., insulin-dependent diabetes and autoimmune diabetes); multiple sclerosis; inflammatory bowel diseases such as ulcerative colitis and Crohn's disease (local enteritis); nontropical sprue, bowel diseases associated with seronegative arthropathy, lymphocytic or collagenic colitis, and diseases associated with leukocyte infiltration into the gastrointestinal tract, such as eosinophilic gastroenteritis; diseases associated with leukocyte infiltration into other epithelial tissues such as the skin, the urinary tract, the respiratory tract, and the joint synovium; pancreatitis; mastitis (mammary gland); hepatitis; cholecystitis; cholangitis or pericholangitis (surrounding tissues of the bile duct and the liver); bronchitis; sinusitis; inflammatory lung diseases causing interstitial fibrosis, such as hypersensitivity pneumonitis; collagen disease; sarcoidosis; osteoporosis; osteoarthrosis; atherosclerosis; neoplasm diseases including neoplasm metastasis or cancerous growth; trauma (trauma healing enhancement); retinal detachment and allergic conjunctivitis; certain kinds of eye diseases such as autoimmune eye disease and uveitis; Sjogren's syndrome; rejection after organ transplantation (chronic and acute); host-versus-graft or graft-versus-host disease; intimal thickening; arteriosclerosis (including graft arteriosclerosis after transplantation); tumor angiogenesis; malignant tumor; multiple myeloma; myeloma-induced bone resorption; central nervous system disorders such as traumatic brain injury and spinal cord injury; and Meniere's disease). In particular, pain can be alleviated in autoimmune diseases such as rheumatoid arthritis (RA) and collagen disease, and arthritis such as knee osteoarthritis, and in particular, pain can be alleviated in the joint of the knee, the shoulder, or the like.

Although the action mechanism through which the expression of TGF-β in plasma is promoted by the oral ingestion of the oral preparation for promoting expression of TGF-β according to this embodiment has not been necessarily clarified, there is a report by the inventors of the invention of the present application that hyaluronic acid binds to a receptor on the surface of the intestinal epithelium in mice having orally ingested the hyaluronic acid (Akira Asari, Tomoyuki Kanemitsu, Hitoshi Kurihara, Oral Administration of High Molecular Weight Hyaluronan (900 KDa) Controls Immune System via Toll-like Receptor 4 in the Intestinal Epithelium). Thus, the action mechanism is estimated as follows: when the hyaluronic acid or the pharmaceutically acceptable salt thereof as the active ingredient of the oral preparation for promoting expression of TGF-β according to this embodiment is orally ingested, the hyaluronic acid or the pharmaceutically acceptable salt thereof binds to a receptor on the surface of the intestinal epithelium, and as a result, the expression of TGF-β in plasma is promoted (see FIG. 10). In addition, it is estimated that a pain suppressing action is exhibited as a result of the promotion of the expression of TGF-β in plasma.

It should be noted that the “patient” in the present invention refers to a human or non-human animal to which the oral preparation for promoting expression of TGF-β according to this embodiment (and an oral preparation for suppressing production of a pain-producing substance and an oral preparation for suppressing edema to be described later) is administered, and generally refers to a human or non-human animal having the above-mentioned disease and/or pain, or a human or non-human animal suspected of the above-mentioned disease and having pain.

The oral preparation for promoting expression of TGF-β according to this embodiment (and an oral preparation for suppressing production of a pain-producing substance and oral preparation for suppressing edema to be described later) is orally administered, and hence a patient does not need to visit a hospital to receive treatment, which can contribute to an improvement in QOL of the patient.

The oral preparation for promoting expression of TGF-β according to this embodiment (and an oral preparation for suppressing production of a pain-producing substance and an oral preparation for suppressing edema to be described later) may be suitably used for the alleviation and/or prevention of pain (inflammatory pain) in the above-mentioned inflammatory disease. For example, the oral preparation for promoting expression of TGF-β according to this embodiment may be used for a patient with mild to severe pain due to the above-mentioned disease.

For example, in the case of the patient with mild pain due to the above-mentioned disease, appropriate treatment for the pain has not been performed in some cases. On the other hand, when the oral preparation for promoting expression of TGF-β according to this embodiment (and an oral preparation for suppressing production of a pain-producing substance and oral preparation for suppressing edema to be described later) is administered to the patient with mild pain due to the above-mentioned disease, pain can be relieved and/or prevented without increasing a burden on the patient.

The oral preparation for promoting expression of TGF-β according to this embodiment (and an oral preparation for suppressing production of a pain-producing substance and oral preparation for suppressing edema to be described later) may contain, in addition to the hyaluronic acid or the pharmaceutically acceptable salt thereof as the active ingredient, other raw materials in such a range that the effects of the present invention are not impaired. Examples of such raw materials include water, an excipient, an antioxidant, a preservative, a wetting agent, a thickener, a buffer, an adsorbent, a solvent, an emulsifier, a stabilizer, a surfactant, a lubricant, a water-soluble polymer, a sweetener, a taste-masking agent, an acidulant, and an alcohol.

No particular limitation is imposed on the dosage form of the oral preparation for promoting expression of TGF-β according to this embodiment (and an oral preparation for suppressing production of a pain-producing substance and an oral preparation for suppressing edema to be described later). When the oral preparation for promoting expression of TGF-β according to this embodiment (and an oral preparation for suppressing production of a pain-producing substance and an oral preparation for suppressing edema to be described later) is orally ingested, examples of the dosage form include preparations for oral administration including: solid preparations such as a tablet, a powder, fine granules, granules, a capsule, and a pill; and liquid preparations such as a solution, a suspension, a syrup, and an emulsion.

The hyaluronic acid or the pharmaceutically acceptable salt thereof is a biological substance, and hence is considered to have no or extremely low side effects even when ingested in a large amount. However, a measure of the amount of the hyaluronic acid and/or the salt thereof to be ingested as the oral preparation for promoting expression of TGF-β according to this embodiment (and an oral preparation for suppressing production of a pain-producing substance and an oral preparation for suppressing edema to be described later) may be from 10 mg to 1,000 mg, preferably from 100 to 500 mg per day. As the number of times of administration, once or a plurality of times per day may be selected depending on symptoms.

### 2. ORAL PREPARATION FOR SUPPRESSING PRODUCTION OF PAIN-PRODUCING SUBSTANCE

An oral preparation for suppressing production of a pain-producing substance according to one embodiment of the present invention contains as an active ingredient hyaluronic acid or a pharmaceutically acceptable salt thereof, and suppresses the production of a pain-producing substance by promoting the expression of TGF-β in plasma. As the hyaluronic acid or the pharmaceutically acceptable salt thereof contained as the active ingredient in the oral preparation for suppressing production of a pain-producing substance according to the one embodiment of the present invention, the hyaluronic acid or the pharmaceutically acceptable salt thereof to be used as the active ingredient in the oral preparation for promoting expression of TGF-β according to the above-mentioned embodiment may be used. In addition, the content and dosage of the hyaluronic acid or the pharmaceutically acceptable salt thereof, and other ingredients in the oral preparation for suppressing production of a pain-producing substance according to the one embodiment of the present invention are also the same as the content and dosage of the hyaluronic acid or the pharmaceutically acceptable salt thereof and other ingredients in the oral preparation for promoting expression of TGF-β according to the above-mentioned embodiment.

For example, when a biological tissue is damaged and inflammation occurs in the tissue, pain is produced. In addition, even after the damaged biological tissue has been repaired, pain is sensed in some cases. In general, bradykinin is known as a typical pain-producing substance. Bradykinin released from plasma at the time of tissue damage excites sensory neurons, thereby producing pain. More specifically, bradykinin has a stimulatory action on a receptor (polymodal receptor) that transmits noxious stimuli to be generated upon tissue damage. That is, bradykinin plays the most important role as a pain-producing substance. Meanwhile, PGE2 is a substance that indirectly exhibits a pain-producing action by the enhancement of the action on the receptor in question by bradykinin. PGE2 is weak in direct pain-producing action compared to bradykinin, but has an action of enhancing pain produced by bradykinin.

A pain-producing substance whose production is suppressed by the oral preparation for suppressing production of a pain-producing substance according to this embodiment may be, for example, bradykinin, or may be both of bradykinin and PGE2. That is, as a result of the suppression of the production of bradykinin by the hyaluronic acid or the pharmaceutically acceptable salt thereof as the active ingredient of the oral preparation for suppressing production of a pain-producing substance according to this embodiment, the production of PGE2 can also be suppressed. As a result, the production of the pain-producing substance can be effectively suppressed.

According to the oral preparation for suppressing production of a pain-producing substance according to this embodiment, the hyaluronic acid or the pharmaceutically acceptable salt thereof promotes the expression of TGF-β in plasma, thereby suppressing the production of a pain-producing substance, and hence inflammatory pain can be relieved. In addition, the oral preparation for suppressing production of a pain-producing substance according to this embodiment is an oral preparation, and hence can be easily ingested by a patient. Thus, the patient does not need to visit a hospital for treatment, and hence a burden on the patient can be reduced, which can contribute to an improvement in QOL of the patient.

### 3. ORAL PREPARATION FOR SUPPRESSING EDEMA

An oral preparation for suppressing edema according to one embodiment of the present invention contains the oral preparation for promoting expression of TGF-3. In the present invention, the “edema” refers to a state in which an excess amount of water (plasma component) accumulates outside blood vessels in a biological tissue. The edema generally develops when a pressure balance between fluid (cellular interstitial fluid) and blood in a cell tissue is lost. Examples of the edema for which the oral preparation for suppressing edema according to this embodiment is administered include local edema and inflammatory edema. Of those, the administration of the oral preparation for suppressing edema according to this embodiment can effectively suppress inflammatory edema, which develops in, for example, the upper extremities (including hands), the lower extremities (including feet), the head, the back, the abdomen, and the hips.

As the hyaluronic acid or the pharmaceutically acceptable salt thereof contained as the active ingredient in the oral preparation for suppressing edema according to this embodiment, the hyaluronic acid or the pharmaceutically acceptable salt thereof to be used as the active ingredient in the oral preparation for promoting expression of TGF-β according to the above-mentioned embodiment may be used. In addition, the content and dosage of the hyaluronic acid or the pharmaceutically acceptable salt thereof, and other ingredients in the oral preparation for suppressing edema according to this embodiment are also the same as the content and dosage of the hyaluronic acid or the pharmaceutically acceptable salt thereof and other ingredients in the oral preparation for promoting expression of TGF-β according to the above-mentioned embodiment.

According to the oral preparation for suppressing edema according to this embodiment, by incorporating the oral preparation for promoting expression of TGF-β, the expression of TGF-β in plasma is promoted through the oral ingestion of the oral preparation for promoting expression of TGF-β. As a result, edema (in particular, edema at the time of inflammation) can be suppressed (see FIG. 10). In addition, the oral preparation for suppressing edema according to this embodiment is an oral preparation, and hence can be easily ingested. Thus, a patient does not need to visit a hospital for treatment, which can contribute to an improvement in QOL of the patient.

### 4. ORAL PREPARATION FOR SUPPRESSING PRODUCTION OF PAIN-PRODUCING SUBSTANCE

4.1. Oral Preparation for Suppressing Production of Pain-Producing Substance

An oral preparation for suppressing production of a pain-producing substance according to one embodiment of the present invention contains as an active ingredient hyaluronic acid or a pharmaceutically acceptable salt thereof, and suppresses the production of a pain-producing substance PGE2 and/or bradykinin.

The average molecular weight of the hyaluronic acid or the pharmaceutically acceptable salt thereof to be used in the oral preparation for suppressing production of a pain-producing substance according to this embodiment is preferably 500,000 or more, more preferably 600,000 or more, still more preferably from 600,000 to 1,600,000. When the average molecular weight of the hyaluronic acid or the pharmaceutically acceptable salt thereof is less than 500,000, it becomes difficult to suppress the production of a pain-producing substance in some cases. In addition, when the average molecular weight of the hyaluronic acid and/or the salt thereof is more than 1,600,000, the hyaluronic acid and/or the salt thereof has difficulty in dissolving, and its effect cannot be sufficiently exhibited in some cases.

The content of the hyaluronic acid or the pharmaceutically acceptable salt thereof in the oral preparation for suppressing production of a pain-producing substance according to this embodiment only needs to be such an amount that the hyaluronic acid or the pharmaceutically acceptable salt thereof can function as the active ingredient, and is generally 1 mass % or more, preferably from 5 to 95 mass %.

4.2. Suppression of Production of Pain-Producing Substance

When the oral preparation for suppressing production of a pain-producing substance according to this embodiment, containing as an active ingredient hyaluronic acid or a pharmaceutically acceptable salt thereof, is orally ingested by humans or non-human animals, the production of a pain-producing substance PGE2 and/or bradykinin is suppressed in cells, tissues, and organs of the humans or the non-human animals.

A pain-producing substance whose production is suppressed by the oral preparation for suppressing production of a pain-producing substance according to this embodiment may be, for example, bradykinin and/or PGE2, and may be one or both of bradykinin and PGE2. For example, as a result of the suppression of the production of bradykinin by the hyaluronic acid or the pharmaceutically acceptable salt thereof as the active ingredient of the oral preparation for suppressing production of a pain-producing substance according to this embodiment, the production of PGE2 can also be suppressed. As a result, the production of the pain-producing substance can be effectively suppressed.

According to the oral preparation for suppressing production of a pain-producing substance according to this embodiment, the hyaluronic acid or the pharmaceutically acceptable salt thereof suppresses the production of the pain-producing substance, thereby making it possible to relieve inflammatory pain. In addition, the oral preparation for suppressing production of a pain-producing substance according to this embodiment is an oral preparation, and hence can be easily ingested by a patient. Thus, the patient does not need to visit a hospital for treatment, and hence a burden on the patient can be reduced, which can contribute to an improvement in QOL of the patient.

When the oral preparation for suppressing production of a pain-producing substance according to this embodiment is orally ingested by humans or non-human animals, the production of the pain-producing substance PGE2 and/or bradykinin can be suppressed in the humans or the non-human animals. As a result of this, pain can be relieved in patients (humans or non-human animals) suffering from inflammatory diseases (e.g., pathological conditions selected from the group consisting of: rheumatoid arthritis (RA); asthma; allergic diseases such as rhinitis; vascular diseases; thrombosis or harmful platelet aggregation; reocclusion after thrombolysis; reperfusion injury; inflammatory skin diseases such as psoriasis, eczema, contact dermatitis, and atopic dermatitis; diabetes (e.g., insulin-dependent diabetes and autoimmune diabetes); multiple sclerosis; inflammatory bowel diseases such as ulcerative colitis and Crohn's disease (local enteritis); nontropical sprue, bowel diseases associated with seronegative arthropathy, lymphocytic or collagenic colitis, and diseases associated with leukocyte infiltration into the gastrointestinal tract, such as eosinophilic gastroenteritis; diseases associated with leukocyte infiltration into other epithelial tissues such as the skin, the urinary tract, the respiratory tract, and the joint synovium; pancreatitis; mastitis (mammary gland); hepatitis; cholecystitis; cholangitis or pericholangitis (surrounding tissues of the bile duct and the liver); bronchitis; sinusitis; inflammatory lung diseases causing interstitial fibrosis, such as hypersensitivity pneumonitis; collagen disease; sarcoidosis; osteoporosis; osteoarthrosis; atherosclerosis; neoplasm diseases including neoplasm metastasis or cancerous growth; trauma (trauma healing enhancement); retinal detachment and allergic conjunctivitis; certain kinds of eye diseases such as autoimmune eye disease and uveitis; Sjogren's syndrome; rejection after organ transplantation (chronic and acute); host-versus-graft or graft-versus-host disease; intimal thickening; arteriosclerosis (including graft arteriosclerosis after transplantation); tumor angiogenesis; malignant tumor; multiple myeloma; myeloma-induced bone resorption; central nervous system disorders such as traumatic brain injury and spinal cord injury; and Meniere's disease). In particular, pain can be alleviated in autoimmune diseases such as rheumatoid arthritis (RA) and collagen disease, and arthritis such as knee osteoarthritis, and in particular, pain can be alleviated in the joint of the knee, the shoulder, or the like.

### 5. ORAL PREPARATION FOR SUPPRESSING EDEMA

An oral preparation for suppressing edema according to one embodiment of the present invention contains as an active ingredient hyaluronic acid or a pharmaceutically acceptable salt thereof, and suppresses the production of a pain-producing substance PGE2 and/or bradykinin. As the hyaluronic acid or the pharmaceutically acceptable salt thereof contained as the active ingredient in the oral preparation for suppressing edema according to this embodiment, the hyaluronic acid or the pharmaceutically acceptable salt thereof to be used as the active ingredient in the oral preparation for suppressing production of a pain-producing substance according to the above-mentioned embodiment may be used. In addition, the content and dosage of the hyaluronic acid or the pharmaceutically acceptable salt thereof, and other ingredients in the oral preparation for suppressing edema according to this embodiment, are also the same as the content and dosage of the hyaluronic acid or the pharmaceutically acceptable salt thereof and other ingredients in the oral preparation for suppressing production of a pain-producing substance according to the above-mentioned embodiment. Examples of the edema for which the oral preparation for suppressing edema according to this embodiment is administered include local edema and inflammatory edema. Of those, the administration of the oral preparation for suppressing edema according to this embodiment can effectively suppress inflammatory edema, which develops in, for example, the upper extremities (including hands), the lower extremities (including feet), the head, the back, the abdomen, and the hips.

As the hyaluronic acid or the pharmaceutically acceptable salt thereof contained as the active ingredient in the oral preparation for suppressing edema according to this embodiment, the hyaluronic acid or the pharmaceutically acceptable salt thereof to be used as the active ingredient in the oral preparation for suppressing production of a pain-producing substance according to the above-mentioned embodiment may be used. In addition, the molecular weight, content, and dosage of the hyaluronic acid or the pharmaceutically acceptable salt thereof, and other ingredients in the oral preparation for suppressing edema according to this embodiment are also the same as the molecular weight, content, and dosage of the hyaluronic acid or the pharmaceutically acceptable salt thereof and other ingredients in the oral preparation for suppressing production of a pain-producing substance according to the above-mentioned embodiment.

According to the oral preparation for suppressing edema according to this embodiment, by incorporating the oral preparation for suppressing production of a pain-producing substance, the production of the pain-producing substance PGE2 and/or bradykinin is suppressed through the oral ingestion of the oral preparation for suppressing production of a pain-producing substance. As a result, edema (in particular, edema at the time of inflammation) can be suppressed. In addition, the oral preparation for suppressing edema according to this embodiment is an oral preparation, and hence can be easily ingested. Thus, a patient does not need to visit a hospital for treatment, which can contribute to an improvement in QOL of the patient.

### 6. EXAMPLES

The present invention is hereinafter described in more detail by way of Examples. However, the present invention is by no means limited to these Examples.

### 6.1. Example 1

### Test for Confirming Pain Suppressing Action of Hyaluronic Acid (In Vivo)

In Example 1, in order to confirm the pain suppressing action of hyaluronic acid and elucidate the action mechanism, an aqueous solution obtained by dissolving hyaluronic acid in distilled water was administered to a rat carrageenan-induced inflammatory pain model by drinking.

6.1.1. Preparation of Test Solution

A test solution was prepared by calculating its concentration based on the average body weight and average drinking amount of rats so that a sample (hyaluronic acid (average molecular weight: 900,000, white powder, manufactured by Kewpie Corporation)) was administered at a dosage of 200 mg/kg/day.

6.1.2. Test Method

Rats (Wistar (SPF), male, four-weeks-old at the time of purchase, purchased from Japan SLC, Inc.) were acclimatized and fed for 7 days, and then grouped based on their body weights on the day before administration. The above-mentioned test solution was administered by drinking ad libitum for 4 weeks. After the administration by drinking for 4 weeks, the rats were selected and grouped so that there was no difference in body weight between groups at the time of carrageenan induction (see Table 1), and then the rats were each used for a pain model to be described later.

TABLE 1

| | | Dosage and | | | Timing of |
|---|---|---|---|---|---|
| | Test | administration period | | | sample |
| Group | substance | of test solution | Induction | n (number) | collection |
| 1 | Distilled | administration of | Absent | 6 | 0 hours after |
| | water | distilled water by | | | induction |
| 2 | (negative | drinking for 4 weeks | Present | 10 | 3 hours after |
| | control) | | | | induction |
| 3 | | | | 10 | 6 hours after |
| | | | | | induction |
| 4 | Test solution | 200 mg/kg/day, | Absent | 6 | 0 hours after |
| | (hyaluronic | administration of test | | | induction |
| 5 | acid aqueous | solution by drinking | Present | 10 | 3 hours after |
| | solution) | for 4 weeks | | | induction |
| 6 | | | | 10 | 6 hours after |
| | | | | | induction |
| 7 | Ibuprofen | 100 mg/kg (10 mg/mL, | Present | 6 | 3 hours after |
| | (positive | 10 mL/kg), oral | | | induction |
| | control) | administration of | | | |
| | | ibuprofen 1 hour before | | | |
| | | induction | | | |

[Rat Pain Model]

A rat pain model was produced by subcutaneously injecting 0.1 mL of a 1% A-carrageenan (manufactured by SIGMA) solution into the plantar surface of the rat hind paw with reference to methods of Ohuchi et al. (Kazuo Ohuchi, “Seibutsu Yakkagaku Jikken Koza (Biological pharmaceutical science experimental course) (Vol. 12) Inflammation and Allergy I-1”, Chapter 1, foreign matter-induced inflammation model: pp. 30-51) and Masahiro Noguchi et al. (Masahiro Noguchi, et al: Enzymologic and pharmacologic profile of loxoprofen sodium and its metabolites. Biol. Pharm. Bull. 2005.28.2075-2079). It should be noted that the 1% λ-carrageenan solution was stirred with a stirrer until immediately before use (induction). After that, a paw volume was measured using a paw volume measuring apparatus. In addition, blood was collected from the rat pain model under isoflurane anesthesia. After that, an exudate was collected from the hind paw, and the measurement of PGE2 and bradykinin in the exudate was performed.

[Positive Control]

The preparation of ibuprofen as a positive control drug and administration conditions therefor are as described below.

Preparation: A required amount of ibuprofen was weighed with an electrobalance and suspended in a 0.5% carboxymethylcellulose (CMC) solution using an agate mortar.
Administration route: Forced oral administration
Administration volume: 10 mL/kg
Administration dose: 100 mg/kg
Number of times of administration: 1 (1 hour before carrageenan administration)
Administration method: Forced oral administration using a 2.5-mL syringe (manufactured by TERUMO CORPORATION) and a soft probe for rats

[Measurement of Paw Volume]

The rat pain model was measured for its paw volume using a paw volume measuring apparatus (PLETHYSMOMETER 101P™ (Muromachi Kikai Co., Ltd.)) before (0 hours after) and 3 hours and 6 hours after carrageenan induction. The tuberal region of the pisiform bone outside the rat right hind paw was marked with marking ink so that volume measurement was uniform. Distilled water was charged up to a target water surface of a measuring chamber of the paw volume measuring apparatus. A measurer restrained the rat, and while lightly pressing the femoral region of the paw to be measured, put the paw at a position at which the mark leveled off and reached the target water surface. Then, a paw volume was recorded using a foot switch connected to the apparatus. It should be noted that the measurement of the paw volume was performed three times for each animal individual, and a mean thereof was determined.

Regarding a measured value for the paw volume, an amount of change Δ mL was determined from the following calculation equation for each individual animal, and a group mean and its standard error (SE) were calculated.

Amount of change A mL=Value at the time of each measurement after carrageenan administration−Value before (0 hours after) carrageenan administration

### [Blood Collection and Plasma Collection]

The rat pain model under anesthesia with isoflurane (manufactured by Mylan Pharmaceuticals, Inc.) was subjected to abdominal section, and blood was collected through the caudal vena cava of the abdomen. A 5-mL disposable injection syringe filled with heparin sodium (Wako Pure Chemical Industries, Ltd.) and a 22-G injection needle were used for the blood collection. Plasma was rapidly separated from the collected blood using a centrifuge, dispensed into a tube, and cryopreserved until use.

(Measurement of TGF-β1 in Plasma)

The measurement of TGF-β1 in plasma was performed using “TGF-β1 Quantikine ELISA Kit (R & D Systems, Inc.).” The operations were performed in conformity with the accompanying protocol of the kit.

(Measurement of IL-10 in Plasma)

The measurement of IL-10 in plasma was performed using “Quantikine Rat IL-10 (R & D Systems, Inc.).” The operations were performed in conformity with the accompanying protocol of the kit.

(Measurement of Concentration of Hyaluronic Acid in Plasma)

The measurement of the concentration of hyaluronic acid in plasma was performed using “Hyaluronan Assay Kit (Seikagaku Biobusiness Corporation).”

[Exudate Collection]

A 15-mL centrifugation tube, which had been measured for its tare weight in advance, was prepared, and a container having a 2-mL pipette tip cut at the tip in the centrifugation tube was prepared. The induced paw of the animal after the end of the blood collection was amputated from 1 cm above the malleolus using bone scissors or the like, and measured for its weight. The skin at the plantar site of the rat was deeply incised using a scalpel to make 2 incisions in a length direction by 4 incisions in a width direction which is perpendicular to the length direction. This paw skin tissue was placed with the toe down into the above-mentioned container, and cooling centrifugation was performed using a centrifugation machine at 3,000 rpm for 15 minutes to collect an exudate. In addition, in a non-treated group, the collection was performed from the left and right hind paws. The mass of the collected exudate was measured using an electrobalance. To the exudate after the end of the mass measurement were added 100 μL of 20 mM aspirin-containing physiological saline and 250 μL of 20 unit/mL heparin-containing physiological saline, the resultant was centrifuged, and then the supernatant was used as an exudate sample. In addition, the exudate was dispensed into a tube. The exudate sample was frozen with liquid nitrogen and cryopreserved until use.

(Measurement of PGE2 in Exudate)

The measurement of PGE2 (ELISA) in the exudate was performed using “Prostaglandin E2 Kit-Monoclonal, ACE.” The operations were performed in conformity with the accompanying protocol of the kit.

(Measurement of Bradykinin in Exudate)

The measurement of bradykinin in the exudate was performed using “Bradykinin, EIA Kit, High Sensitivity, Bachem Americans.” The operations were performed in conformity with the accompanying protocol of the kit.

6.1.3. Test Results

[Paw Edema]

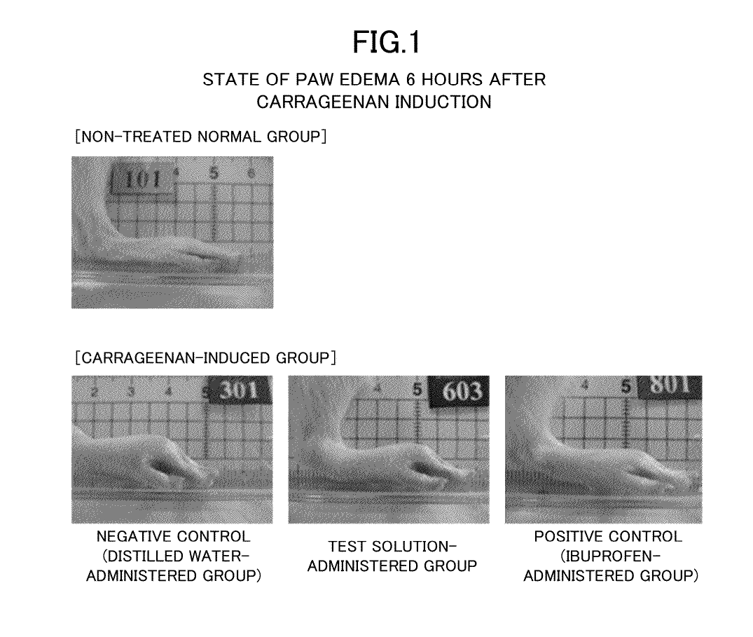

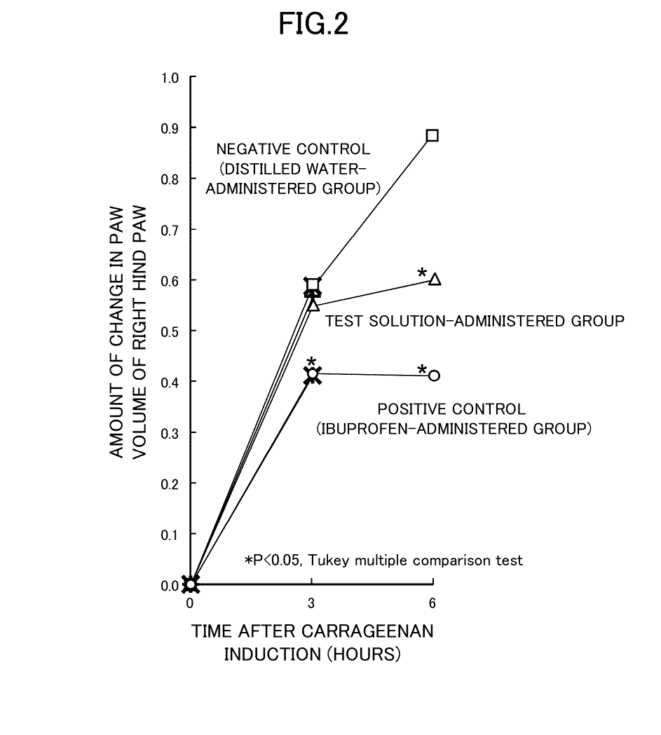

In the negative control (distilled water-administered group), an increase in paw edema was observed with the lapse of time (see FIG. 2). In the test solution (hyaluronic acid aqueous solution)—administered group and the positive control (ibuprofen-administered group), edema was milder than in the negative control 6 hours after carrageenan induction (see FIG. 1).

[Paw Volume]

In the negative control (distilled water) group, an increase in paw volume was observed with the lapse of time (see FIG. 2). It should be noted that in FIG. 2, the ordinate axis indicates, as an amount of change in paw volume of the left hind paw, a paw volume relative to a paw volume before carrageenan induction. In the test solution (hyaluronic acid aqueous solution)—administered group, the paw volume 6 hours after carrageenan induction was significantly smaller than that in the negative control. In the positive control (ibuprofen-administered) group, the paw volumes 3 hours and 6 hours after carrageenan induction were significantly smaller than those in the negative control.

[Exudate Amount]

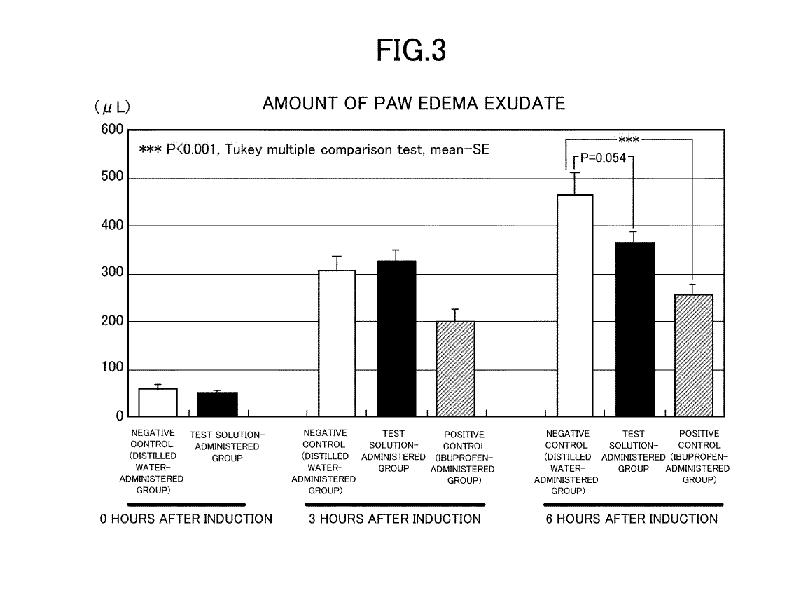

As shown in FIG. 3, the exudate amount of the paw before and after carrageenan induction showed almost the same tendency as the paw volume. That is, in the negative control (distilled water) group, an increase in exudate amount of the paw was observed with the lapse of time (see FIG. 3). On the other hand, in the test solution (hyaluronic acid aqueous solution)—administered group, the exudate amount of the paw 6 hours after carrageenan induction was smaller than that in the negative control. In addition, in the positive control (ibuprofen-administered) group, the exudate amounts of the paw 3 hours and 6 hours after carrageenan induction were significantly smaller than those in the negative control.

[Concentration of TGF-β in Plasma]

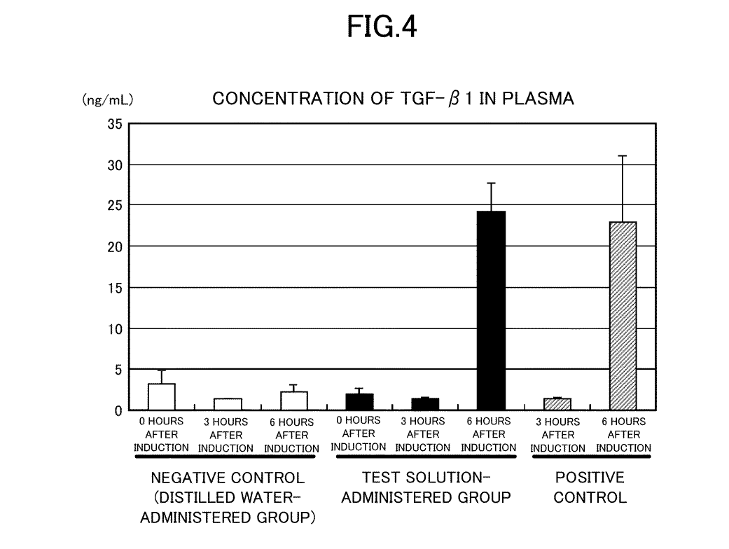

It was confirmed that the concentration of TGF-β in plasma in the test solution (hyaluronic acid aqueous solution)—administered group increased remarkably 6 hours after carrageenan induction, which was higher than the concentration of TGF-β in the positive control (ibuprofen) at the same timing (see FIG. 4). On the other hand, the concentrations of TGF-β in plasma in the negative control (distilled water-administered group) 3 hours and 6 hours after carrageenan induction showed almost no change compared to that immediately after carrageenan induction. Thus, it was confirmed that the production of TGF-β in plasma was promoted by the oral administration of hyaluronic acid.

[Bradykinin in Exudate]

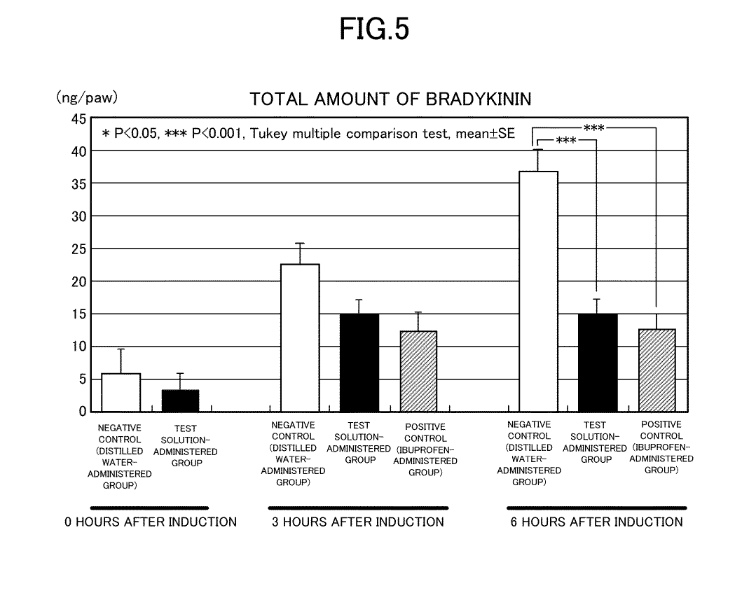

In the negative control (distilled water-administered group), an increase in total amount of bradykinin was observed with the lapse of time (see FIG. 5). In the test solution (hyaluronic acid aqueous solution) —administered group, the total amount of bradykinin 6 hours after carrageenan induction was significantly smaller than that in the negative control. In the positive control (Ibuprofen-administered group), the total amounts 3 hours and 6 hours after carrageenan induction were smaller than those in the negative control (statistically significant 6 hours after carrageenan induction).

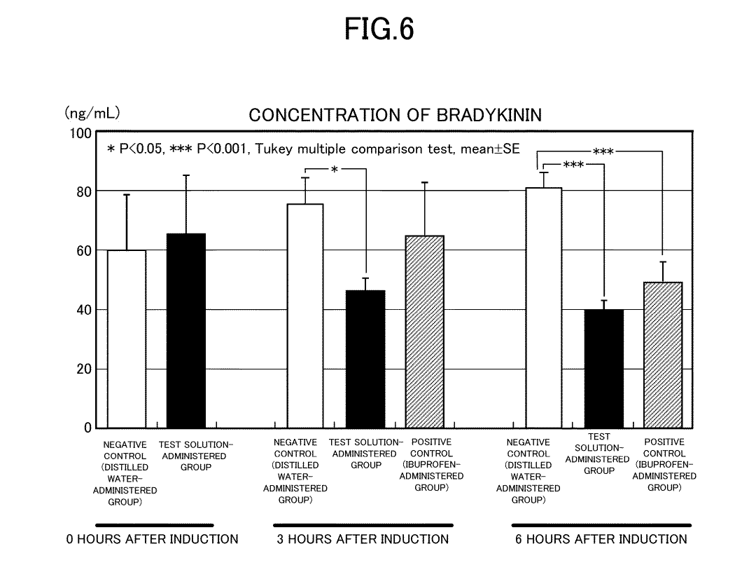

In the negative control, an increase in concentration of bradykinin was observed with the lapse of time (see FIG. 6). In the test solution-administered group, it was confirmed that the concentrations of bradykinin 3 hours and 6 hours after carrageenan induction were both significantly smaller than those in the distilled water group, and the concentrations of bradykinin were lower than those in the positive control. In the positive control, a significantly smaller concentration of bradykinin than that in the negative control was confirmed only 6 hours after carrageenan induction.

[PGE2 in Exudate]

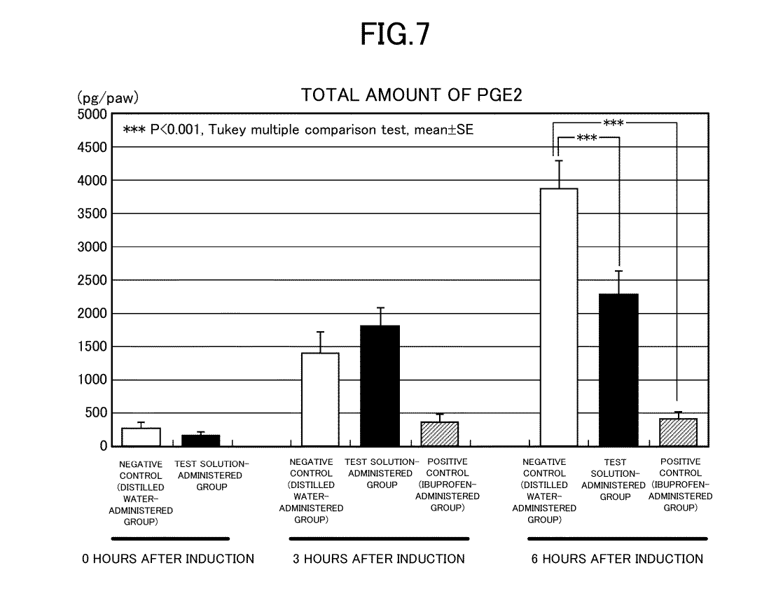

The total amount of PGE2 in the exudate was found to increase with the lapse of time in the negative control (distilled water-administered group) (see FIG. 7). In the test solution (hyaluronic acid aqueous solution)—administered group, the total amount of PGE2 6 hours after carrageenan induction was found to be significantly smaller than that in the negative control. In the positive control (ibuprofen-administered group), the total amounts of PGE2 3 hours and 6 hours after carrageenan induction were smaller than those in the negative control (statistically significant 6 hours after carrageenan induction).

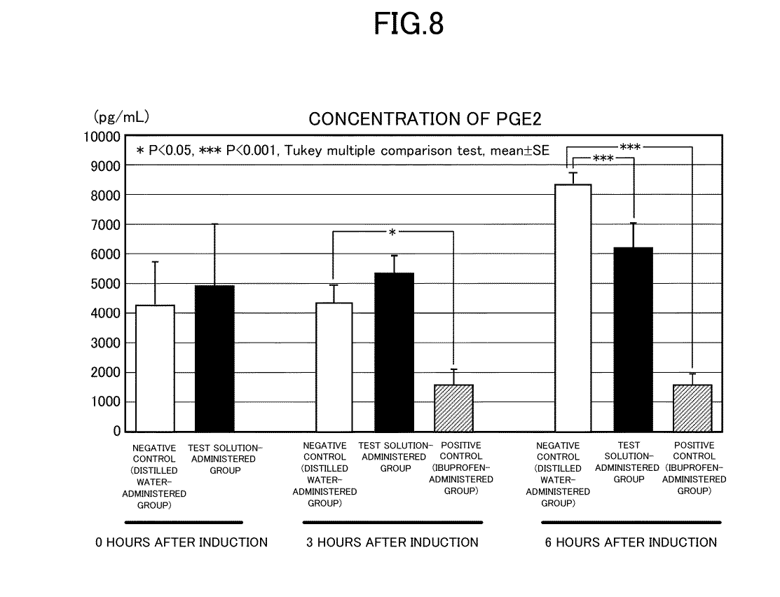

In the negative control, no difference in concentration of PGE2 was found between immediately after induction and 3 hours after induction. On the other hand, in comparison between 3 hours and 6 hours after carrageenan induction, an increase in concentration of PGE2 was observed with the lapse of time (see FIG. 8). In the test solution-administered group, the concentration of PGE2 6 hours after carrageenan induction was significantly smaller than that in the negative control. In the positive control, the concentrations of PGE2 were significantly smaller than those in the negative control 3 hours and 6 hours after carrageenan induction.

[Concentration of Hyaluronic Acid in Plasma]

The concentrations of hyaluronic acid in plasma of the negative control (distilled water-administered group) and the test solution-administered group were measured. The concentrations of hyaluronic acid in plasma were measured before carrageenan induction and 6 hours and 8 hours after carrageenan induction.

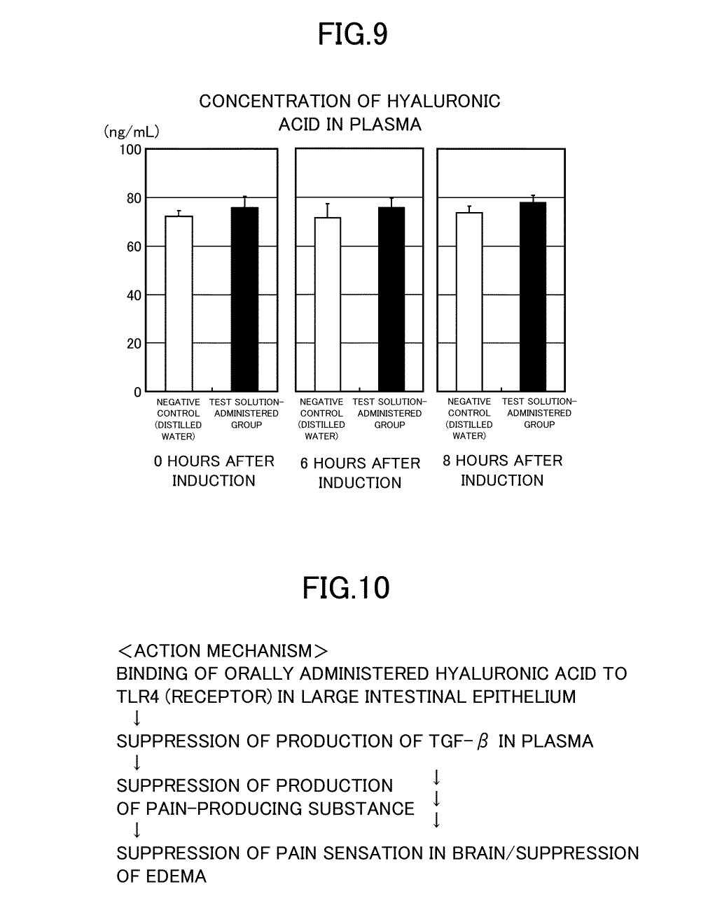

As shown in FIG. 9, in both of the negative control (distilled water-administered group) and the test solution (hyaluronic acid aqueous solution) —administered group, the continuous ingestion of the hyaluronic acid for 4 weeks did not cause any change in concentration of the hyaluronic acid in plasma (see the left panel of FIG. 9). In addition, in both of the distilled water-administered group and the test solution-administered group, almost no change in concentration of the hyaluronic acid in plasma was found 6 hours and 8 hours after carrageenan induction (see the central panel and right panel of FIG. 9). Thus, it can be said that irrespective of the presence or absence of the ingestion of hyaluronic acid, the carrageenan induction had no influence on the concentration of the hyaluronic acid in plasma. The results indicate that the hyaluronic acid in plasma is not very deeply involved in the promotion of the expression of TGF-β in plasma.

6.1.4. Discussion

As shown in FIG. 1, the administration of the test solution (hyaluronic acid aqueous solution) was found to suppress carrageenan-induced paw edema. In addition, as shown in FIG. 5, FIG. 6, FIG. 7, and FIG. 8, the administration of the test solution (hyaluronic acid aqueous solution) was found to suppress the production of bradykinin and PGE2 in the exudate of the paw. Thus, it is considered that the suppression of paw edema is a change associated with the suppression of the production of bradykinin in the paw and the suppression of the production of PGE2 due to the suppression of the production of bradykinin.

In particular, the concentration of bradykinin in the exudate of the paw showed a lower value in the test solution (hyaluronic acid aqueous solution)—administered group than in the positive control (ibuprofen group). Thus, the oral administration of hyaluronic acid was found to be able to effectively suppress the production of bradykinin.

In addition, an increase in IL-10 in plasma was not observed.

This suggests that the oral administration of hyaluronic acid directly suppressed the production of TGF-β.

### 6.2. Example 2

### TGF-β Expression Promoting Action of Hyaluronic Acid in HT29 Cells (In Vitro)

In order to confirm the TGF-β expression promoting action of hyaluronic acid in human colon adenocarcinoma-derived HT29 cells (purchased from DS Pharma Biomedical Co., Ltd.), the measurement of the production amount of TGF-β mRNA in the case of treating HT29 cells with hyaluronic acid was performed by the following method.

Hyaluronic acid was added to a culture supernatant, and 1 hour after that, a lipopolysaccharide (LPS) was added at a concentration of 200 ng/mL. 23 hours after that, mRNA of cells was collected, and the determination of TGF-β1 mRNA using real-time PCR equipment (Mx 3005, Agilent Technologies) was performed. First, cDNA was synthesized from mRNA by reverse transcription, and a PCR reaction was performed using the cDNA. That is, an amplification reaction involving repeating thermal denaturation (95° C., 10 seconds) and annealing (60° C., 20 seconds) 40 times was performed, and simultaneously, DNA amplification was measured by monitoring fluorescence from CybrGreen to be conjugated with DNA, and determination in the case of defining the mRNA amount of a control as 1 was performed. The human TGF-31 primers used were designed with the software Primer BLAST made available to the public by the National Center for Biotechnology Information (NCBI). Their sequences are 5′-TTCGCCTTAGCGCCCACTGC-3′ (Forward) and 5′-CAGGGCCAGGACCTTGCTGTACT-3′ (Reverse). The GAPDH primers were designed in conformity with the literature of Asari et al. (Akira Asari, Tomoyuki Kanemitsu, Hitoshi Kurihara, Oral Administration of High Molecular Weight Hyaluronan (900 KDa) Controls Immune System via Toll-like Receptor 4 in the Intestinal Epithelium). Their sequences are 5′-ACCACAGTCCATCAC-3′ (Forward) and 5′-TCCACCACCCTGTTGCTGTA-3′ (Reverse).

The average molecular weights of the hyaluronic acids used are 8,000 (8 k), 50,000 (50 k), and 800,000 (800 k) (each of which is manufactured by Kewpie Corporation), respectively. A hyaluronic acid aqueous solution prepared by dissolving each hyaluronic acid in water so that the concentration of each hyaluronic acid was 0.01 mg/ml was used.

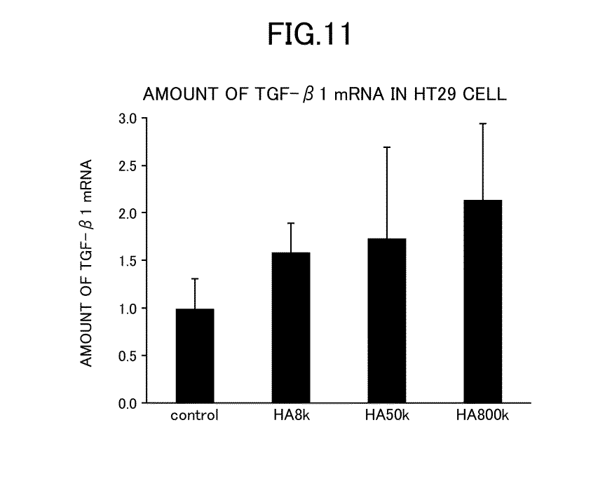

FIG. 11 shows the results. It should be noted that in FIG. 11, the production amount of TGF-β1 mRNA in the case of treatment with each hyaluronic acid is shown as a relative amount with respect to the production amount of TGF-β1 mRNA in the case of no addition of hyaluronic acid (control). It should be noted that control, HA8k, HA50k, and HA800k in FIG. 11 mean the control, the hyaluronic acid having a molecular weight of 8,000, the hyaluronic acid having a molecular weight of 50,000, and the hyaluronic acid having a molecular weight of 800,000, respectively.

As shown in FIG. 11, the amount of TGF-β1 mRNA in HT 29 cells 24 hours after addition of the hyaluronic acid was found to increase as the molecular weight of the hyaluronic acid increased. The amount of TGF-β1 mRNA in the hyaluronic acid having a molecular weight of 800,000 was 2.14 times as high as that in the control.

### 6.3. Example 3

The hyaluronic acid used in Example 1 (hyaluronic acid having an average molecular weight of 900,000) was used as the oral preparation for promoting expression of TGF-β (or the oral preparation for suppressing production of a pain-producing substance, or the oral preparation for suppressing edema) to produce a soft capsule in which the contents were blended as described below.

[Blending Ratio]

| | |
|---|---|
| Oral preparation for promoting expression of TGF-β (hyaluronic | 20% |
| acid of Example 1) | |
| Olive oil | 50% |
| Beeswax | 10% |
| Medium chain fatty acid triglyceride | 10% |
| Emulsifier | 10% |
| | 100% |

### 6.4. Example 4

The hyaluronic acid used in Example 1 (hyaluronic acid having an average molecular weight of 900,000) was used as the oral preparation for promoting expression of TGF-β (or the oral preparation for suppressing production of a pain-producing substance, or the oral preparation for suppressing edema) to produce a powder (granule) blended as described below.

[Blending Ratio]

| | |
|---|---|
| Oral preparation for promoting expression of TGF-β (hyaluronic | 10% |
| acid of Example 1) | |
| Lactose | 60% |
| Corn starch | 25% |
| Hypromellose | 5% |
| | 100% |

### 6.5. Example 5

The hyaluronic acid used in Example 1 (hyaluronic acid having an average molecular weight of 900,000) was used as the oral preparation for promoting expression of TGF-β (or the oral preparation for suppressing production of a pain-producing substance, or the oral preparation for suppressing edema) to produce a tablet blended as described below.

[Blending Ratio]

| | |
|---|---|
| Oral preparation for promoting expression of TGF-β (hyaluronic | 25% |
| acid of Example 1) | |
| Lactose | 24% |
| Crystalline cellulose | 20% |
| Corn starch | 15% |
| Dextrin | 10% |
| Emulsifier | 5% |
| Silicon dioxide | 1% |
| | 100% |

1. An oral preparation for promoting expression of TGF-β, comprising as an active ingredient hyaluronic acid or a pharmaceutically acceptable salt thereof, the oral preparation promoting expression of TGF-β in plasma.

2. An oral preparation for suppressing production of a pain-producing substance, comprising as an active ingredient hyaluronic acid or a pharmaceutically acceptable salt thereof, the oral preparation suppressing production of a pain-producing substance by promoting expression of TGF-β in plasma.

3. The oral preparation for suppressing production of a pain-producing substance according to claim 2, wherein the pain-producing substance comprises bradykinin.

4. An oral preparation for suppressing edema, comprising the oral preparation for promoting expression of TGF-β according to claim 1.

5. An oral preparation for suppressing production of a pain-producing substance, comprising as an active ingredient hyaluronic acid or a pharmaceutically acceptable salt thereof, the oral preparation suppressing production of a pain-producing substance PGE2 and/or bradykinin.

6. An oral preparation for suppressing edema, comprising as an active ingredient hyaluronic acid or a pharmaceutically acceptable salt thereof, the oral preparation suppressing production of a pain-producing substance PGE2 and/or bradykinin.


SEQUENCE LISTING (4 sequences)

SEQ ID NO: 1 - DNA, 20 bp, Artificial sequence
  Primer
ttcgccttagcgcccactgc

SEQ ID NO: 2 - DNA, 23 bp, Artificial sequence
  Primer
cagggccaggaccttgctgtact

SEQ ID NO: 3 - DNA, 15 bp, Artificial sequence
  Primer
accacagtccatcac

SEQ ID NO: 4 - DNA, 20 bp, Artificial sequence
  Primer
tccaccaccctgttgctgta